(19) United States
(12) Patent Application Publication
(10) Pub. No.: US 2017/0234855 A1
(43) Pub. Date: Aug. 17, 2017

(54) MINITURIZED HIGH EFFICIENCY SCREENING PROCESS FOR IDENTIFYING FUNGAL EXTRACTS WITH POTENTIAL THERAPEUTIC VALUE

(71) Applicant: University of Findlay, Findlay, OH (US)

(72) Inventor: Michael Edelbrock, Findlay, OH (US)

(73) Assignee: University of Findlay, Findlay, OH (US)

(21) Appl. No.: 15/403,750

(22) Filed: Jan. 11, 2017

Related U.S. Application Data
(60) Provisional application No. 62/277,591, filed on Jan. 12, 2016.

(51) Int. Cl. G01N 33/50; C12Q 1/68

(52) U.S. Cl. CPC G01N 33/5011; C12Q 1/6895; G01N 33/5014; G01N 2333/37

(57) ABSTRACT

A high throughput, micro-screening process for the identification of fungal extracts that contain secondary metabolites with bioactivity is disclosed.

### CROSS REFERENCE TO RELATED APPLICATION

The present patent application is based upon and claims the benefit of provisional patent application No. 62/277,591, filed on Jan. 12, 2016.

### BACKGROUND OF THE INVENTION

The use of plants and fungi to produce novel compounds is well documented. Natural products have accounted for an estimated 40% of prescription-based drugs in the United States. Specifically, fungi are recognized as extraordinary producers of unique metabolites and compounds. They can enzymatically create complex molecules that traditional synthetic organic chemistry is incapable of producing. Fungi represent an untapped area, of biological organisms capable of producing chemically and structurally complex compounds with previously unknown bioactivity against a variety of human cancers and pathogenic microorganisms. For example, fungal terpenes have documented bioactivity against several human cancers, antitumor activity, (Fulda and Kroemer, 2009; Saleem et al., 2009) anti-HIV properties (Barroso-González et al., 2009; Qian et al., 2009), activity against the herpes simplex virus (Badria et al., 2003) and Epstein-Barr virus (Nakagawa-Goto et al., 2009). Anticancer and antitumor activity of the fungus Omphalotus illudens is currently under phase II trials against prostate and ovarian cancers (McMorris et al., 2007, Yeo et al., 2007). Species of marine, insect-associated, and mycophilic fungi seem to be promising groups that possess bioactivity and are largely understudied (Schneider et al., 2008).

### SUMMARY OF THE INVENTION

The present invention is a high throughput, micro-screening process for the identification of fungal extracts that contain secondary metabolites with bioactivity. We have, after collection of the fungus, developed a new process to micronize and identify fungal extracts which contain a bioactive compound. Each individual step in the process and the components used have been developed to work in a small-scale format. The instrumentation and reagents have been assembled in such a manner as to increase productivity and speed at each process step while reducing cost. From early discovery to present, researchers have relied on past publications to follow methods involving large scale methods to generate fungal extracts. As much as 3 to 5 kilograms of fungus from 1-2 liters large broth solutions were used to identify novel compounds or conduct bioactivity experiments. These conditions require 3 weeks to generate the actual culture conditions that have been used to conduct functional activity studies with the same outcomes of our process. (In-Kyoung Lee, et al. 2007), (Ki Hyun Kim, et al., 2007), (Suthep Wiyarutta, et al., 2004), (Priyani A. Paranagama, et al., 2007), (Jixun Zhan, et al., 2007). Indeed, these methods may yield results that seemingly have cytotoxic activity when in fact this may be an artifact. Large extracts which are reduced to low volume may show promise due to a higher concentration of a compound of limited bioactivity. This will in fact slow down the discovery and identification process while red herrings are produced and investigated due to large scale methods.

Conversely, a process for miniaturizing an extraction protocol has been developed for analysis of fungal produced chemicals. (Bragulat, et al., 2001), (Nielsen 2003), (Smedsgaard 1997) However, in each of these cases, the goal was for determination of the presence of a particular chemical compound(s) using instrumentation sensitive to detecting trace levels. The identification of trace levels of compounds of potential bioactivity does not address whether the detected compounds actually have bioactivity. Indeed, the compounds investigated for their presence in fungal extracts have often been previously determined to have activity by the large-scale extractions. Further strengthening the need for a more efficient and faster process which relies on less manpower to achieve the same results. In our process, we take advantage of the limitations in each method to develop a new process to determine bioactivity. Our process minimizes the actual extraction process to generate enough extract to conduct sensitive bioactivity studies of fungal metabolites while reducing the potential of false results. This process is important as it can generate natural compounds with potential anticancer properties in a faster more sensitive screening method. Thereby, quickening the discovery and identification of novel compounds which may have greater efficacy in chemotherapeutics.

Other objectives and advantages of the present inversion will become apparent to those skilled in the art upon a review of the following detailed description of the preferred embodiments and the accompanying drawings.

### BRIEF DESCRIPTION OF THE DRAWINGS

FIG. 1 is a flow chart showing the process of the present invention.

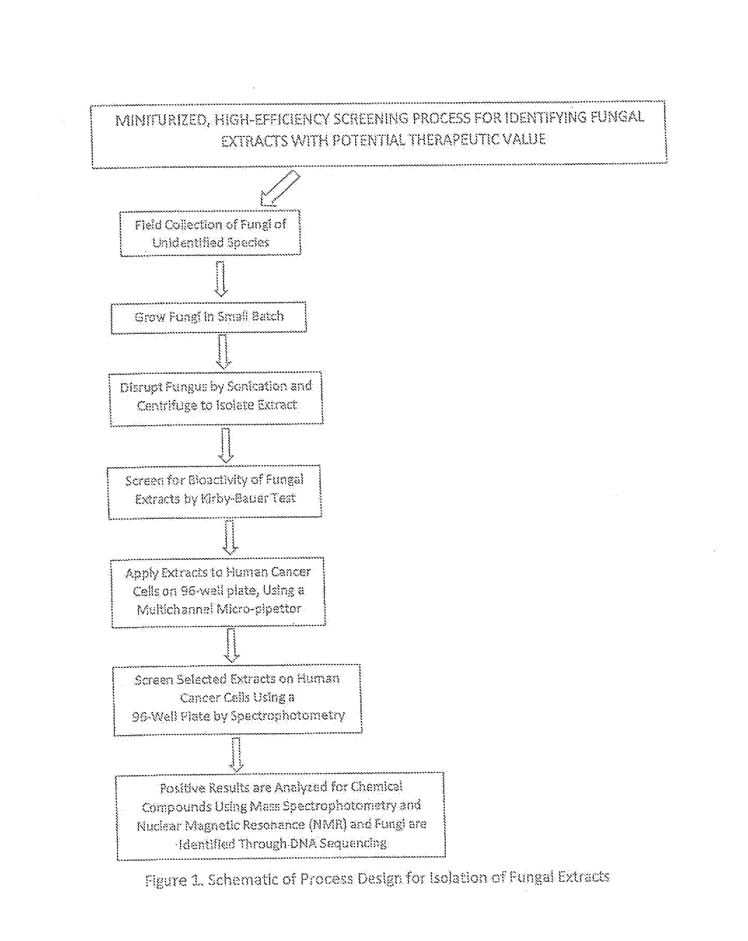

### DETAILED DESCRIPTION OF THE PREFERRED EMBODIMENTS

The invention is a high throughput miniaturized screening process for the identification of fungal extracts that contain secondary metabolites with bioactivity. The features of the invention will be more readily understood by referring to the drawings in connection with the following description.

Sample Collection: Fungal samples are collected from various substrates including soil, vegetation and animal flora. A high number of different fungal species are collected which will be needed for the basis of a high throughput miniaturized strategy. In the proposed model the ability of spore collection will be an important aspect to the project and will involve collection trips to several geographical areas to obtain spores of a diverse range of species. Upon fungal collection, serial dilutions are performed to isolate individual colony forming units (spores/hyphae) for generating pure cultures. Fungal species will be grown onto pure culture of corn meal agar (Difco) containing the antibiotics tetracycline, erythromycin, penicillin G, streptomycin, ampicillin (aqueous solution of 0.025 g/25 mL each). Pure cultures will be grown on potato dextrose agar (PDA) for one week at room temperature under 24-hour fluorescent lighting. To identify species of fungi of interest, the internal transcribed spacer region of rDNA (ITS) are sequenced using the following methodology. Cultures are grown on PDA plates for five days and a 1 cm² scraping of mycelium placed into a 2 ml microcentrifuge tube with silica beads. DNA is extracted using a Qiagen Puregene Core Kit A or comparable technology according to the manufacturers instructions after grinding the mycelium with a plastic pestle and vortexing for 10 minutes. PCR is conducted on an Eppendorf Mastercycler or comparable instrument. PCR reactions are then purified using ExoSapIT according to the manufacturer's instructions. DNA sequencing will be conducted at the Chicago CRC DNA sequencing facility or other institution recognized as proficient by the scientific community. Species identification will then be accomplished using the UNITE ver. 6.0 database or comparable software (Köljalg et al. 2013) with a cut-off value of 1.5% identity to the closest species hypothesis. The sequencing of DNA for identification purposes is a high throughput method in and of itself and is independent of our novel process.

After one week of growth on FDA plates as previously described, pure cultures are cut into many small squares and then inoculated into 40 milliliters potato dextrose broth (Difco) in Erlenmeyer flasks. Liquid cultures are grown for 5 days at 24° C. in a shaker incubator set at 180 rpm. These volumes are dramatically reduced (from 500-1000 ml down to 40 milliliters) and time dramatically reduced (5 days compared to 21 days). The medium and cells are transferred in to 50 mL screwcap tubes. The cells of the mycelium are disrupted by sonication at 75% power for 60 minutes. Largescale protocols rely upon blending and then filtration. Our method eliminates the need for filtration, since the smaller volume can be easily centrifuged while larger containers cannot. Our resulting slurry is then centrifuged at high speed (14,000 rpm) for 5 minutes and the aqueous phase removed. The secondary metabolites are extracted from the solution by solid-liquid partitioning using 10 milliliters of ethyl acetate. Compared to older high volume extractions this step reduces the volume and cost of organic solvents by a 10-fold reduction (10 milliliter compared to 100 milliliters). Additionally, some older methods call for the use of chloroform or methanol or a combination of both. These extraction solvents are volatile and residues must be handled as hazardous waste. Both solvents are poisonous and chloroform is a listed carcinogen while methanol is highly flammable. We have reduced both health and environmental concerns at a reduced cost. Researchers investigating compounds at sensitive detection would be able to easily use a fraction from our process. A simple syringe filter would remove particles that may interfere with their analysis. Thereby eliminating the need for the extraction to be conducted twice. The organic phase containing the metabolites are then separated from the aqueous phase. Upon solvent extraction, one milliliter of extract is removed and dried using an evaporation centrifuge. Samples are dried and resuspended in 100 uL of dimethyl sulfoxide (DMSO). Several aliquots are generated and the concentrated extract is used for several experimental treatments. Vehicle controls are made side by side with samples for quality control purposes.

Fungal extracts of 100 uL volume will be used to screen using standardized conditions with the Kirby-Bauer Disc Diffusion method for activity against (Saccharomyces cerevisiae) yeast. PDA (15 ml) are poured into 90 mm Petri plates and 8-10 autoclaved filter paper discs (6 mm) placed aseptically onto the agar surface. Raw extract (30-100 μl) are dispensed onto each filter paper disc and cultures incubated for 24-48 hours and diameter of the zone of inhibition recorded (if present). Fungal extracts with bioactivity against yeast cells are selected for screening using human cell lines.

Human cells will include but are not limited to HCT116 (human colon cancer), HeLa (human cervical carcinoma) and/or SK-Mel28 (human melanoma) are cultured in Iscove's Modified Dulbecco's Media (IMDM) supplemented with 10% fetal bovine serum (FBS) or other comparable media recognized by the American Type Culture Collection (ATCC). Cells are incubated in a humidified chamber at 37° C. 5% CO₂. Cells will be grown to 70-80% confluence during normal culturing.

Cells are uniformly plated at 2,500 cells per well with 200 uL of cell culture media onto a 96-well plate format. Then are incubated for 24 hours to allow for cell adhesion. Control and treatment plates are prepared in triplicate. Experimental plates are treated with 1 uL of a diluted extract 1/10 in media or an appropriate dilution determined to be effective in screening extracts in a dose dependent manner. Control plates will receive 1.0 uL DMSO at the same dilution as treatment plates. Plates are incubated for an additional 2-3 days, the media removed and plates rinsed with refrigerated phosphate buffered saline (PBS). A commercially available kit is used to determine the viability of cells by spectrophotometry using the manufacturer's protocol. The use of a 96-well format for cell viability measurements is a well-accepted State-of-Art technique. However, not all labs are equipped to use this technique and still rely on more costly techniques. The specific instrument, equipment, and reagents envisioned include but are not limited to the following:

### Molecular Devices Inc

A Flex-3 or i3x spectrophotographic plate reader 89212-402
SoftMax Pro Software included with spectrophotometer
Viability/Cytotoxicity Assay Kit for Animal Live and Dead Cells 89138-886
Calcein-AM Cell Viability Assay Kit 89138-954

### Cell Bio Labs

OxiSelect™ DNA Double-Strand Break (DSB) Staining Kit STA-321

### VWR

Cell integrity kit item 10127-642
Assorted supplies, growth media, culture plates, fetal calf serum, tissue culture supplies commonly found in molecular labs

Upon screening of fungal extracts by one or more of the above referenced techniques bioactive spores are isolated for further characterization. Verification is done as appropriate using serial dilutions. The actual bioactive compound is unknown within the extract and the definition of cytotoxic is subjective to the process. We use complete toxicity at a 1 uL treatment with an extract at a 100 fold dilution as our determination of high cytotoxicity. Those extracts exhibiting cytotoxicity are further diluted and their chemical composition determined by standard chemical analysis techniques independent of the scope of our novel process.

We estimate an approximate savings of $20,000 to process 1000 extracts including control samples in triplicate or 3000 samples (Table 1).

Currently used Large-Scale Process

| | | Large- |
|---|---|---|
| Item | Cost | Scale |
| Culture Plates, | 1.575 each | 4,725.00 |
| 3000 | | |
| FBS, 3,600 mL | 1.65/mL | 5,940.00 |
| IMDM Media, | $0.03/mL | 1,080.00 |
| 36000 mL | | |
| Penn/Strep, 360 mL | $1.2/mL | 432.00 |
| MicroPipette tips, | $180/1000 | 540.00 |
| 3000 | | |
| Pipette 10 ml, 300 | $230/200 | 345.00 |
| Pipette 25 ml, 100 | $212/200 | 106.00 |
| Ethyl Acetate, 100 L | $78.75/L | 7,875.00 |
| Potatose Dextrose | $6/L | 3,000.00 |
| Broth 500 L | | |
| Methanol, 5 L | $48/L | 240.00 |
| Isopropanol, 1 L | $53/L | 53.00 |
| Crystal Violet, 25g | $95/25 g | 95.00 |
| | Total | 24,421.00 |

Novel Micro-Scale Process

| | | Micro- |
|---|---|---|
| Item | Cost | Scale |
| 96 well plates | 4.2 each | 126.00 |
| (30) | | |
| FBS, 60 mL | 1.65/mL | 99.00 |
| IMDM Media, | $0.03/mL | 18.00 |
| 600 mL | | |
| Penn/Strep, 6 ml. | $1.2/ml | 7.20 |
| MicroPipette | $180/1000 | 540.00 |
| tips, 10 uL 3000 | | |
| MicroPipette | $180/1000 | $540.00 |
| tips, 300 uL, 3000 | | |
| Falcon tubes, | $370/500 | $720.00 |
| 50 ml., 1000 | | |
| Ethyl Acetate 10 L | $78.75/L | $787.50 |
| Potatose | $6/L | $240.00 |
| Dextrose | | |
| Broth, 40 L | | |
| 96 Well Assay | $350/1K | $1,050.00 |
| Kits 3K assays | | |
| | Total | $4,127.00 |

The manpower costs are hard to determine. In older methods, technicians count viable colonies under the microscope at 5 minutes per plate or 250 hours to complete 3000 plates. The automated plate reader can scan and analyze a plate of 96 samples in 10 minutes or approximately 5 hours to complete the analysis of 3000 samples.

Many cancers acquire multidrug resistance and this is a growing problem. The discovery of new chemotherapeutic drugs is crucial to counter multidrug resistance. To this end, biological organisms have clearly been identified as potential sources of novel compounds with therapeutic properties. We have designed a rapid and novel process for drug discovery of fungal metabolites that have bioactivity against cancer cells. The older techniques to generate fungal extracts have lagged behind advances in technology and instrumentation designed for rapid screening of bioactive compounds. Our unique process takes advantage of improvements in technology combined with a newly designed method to generate and test fungal metabolites. Our process is a miniaturized version of older, classic molecular biological techniques combined with sensitive technology. This provides a rapid and efficient approach greatly needed to quickly search for novel anticancer drugs. Simultaneously we have dramatically saved time, money and manpower. Our process reduces potential health complications of toxic chemicals and dramatically reduces hazardous waste generation and toxic air emissions making it a powerful tool for rapid drug discovery.

The above detailed description of the present innovative process is given for explanatory purposes. It will be apparent to those skilled in molecular biology techniques that changes and modifications can be made without departing from the scope of the invention. Our results have demonstrated that this process can be used to minimized cost. Accordingly, the whole of the foregoing description is to be construed in an illustrative and not a limitative sense, the scope of the invention being defined solely by the appended claims.

We claim:

1. Process for identifying fungal extracts with potential therapeutic value comprising:
growing isolated fungal species at micro level from fungi of unidentified species;
extracting secondary metabolites from the fungal species using a solvent;
screening for bioactivity using fungal species;
identifying fungi with bioactivity and bioactivity on human cells;
selecting human cells and treating the human cells with extracts from the fungi;
selecting extracts of fungi with high bioactivity and growing the extracts in a large culture;
extracting DNA from the fungi extracts to identify species.

2. The process of claim 1 in which the initial screen for bioactivity is down by the Kirby-Bauer technique.

3. The process of claim 1 in which the selected human cells are treated on a 96-well culture dish.
